(19) United States
(12) Patent Application Publication
(10) Pub. No.: US 2016/0098533 A1
(43) Pub. Date: Apr. 7, 2016

(54) System and Method for Improving Health Care Management and Compliance

(71) Applicant: AlignCare Services, LLC., New York, NY (US)

(72) Inventors: Stephen Jackson, New York, NY (US); Manon Ruben, New York, NY (US); William McCann, New York, NY (US)

(21) Appl. No.: 14/877,702

(22) Filed: Oct. 7, 2015

Related U.S. Application Data
(60) Provisional application No. 62/060,813, filed on Oct. 7, 2014.

(51) Int. Cl. G06F 19/00; G06F 17/30; H04L 29/08

(52) U.S. Cl. CPC G06F 19/3443; G06F 19/3456; G06F 19/3431; H04L 67/06; G06F 17/30598

(57) ABSTRACT

A computer implemented system and method for quantifying a risk associated with medical and health care, the steps of which have; calculating, via a processor, a first value; the first value equal to the medications prescribed to a plurality of individuals in a specified population; calculating, via a processor, a second value; the second value equal to the prescribers of the first value of the plurality of individuals in a specified population; calculating, via a processor, a third value; the third value equal to the associated therapeutic classifications of the first value of the plurality of individuals in a specified population; calculating, via a processor, an average value of each first, second and third value; comparing, via a processor, the average value of each first, second and third value against a fourth value; the fourth value equal to a single individual of the first, second and third value; transforming, via a processor, the first, second, third and fourth values into a fifth value, calculating, via a processor, the fifth value which corresponds to a degree of risk of adverse outcomes related to the healthcare management of an individual.

### CLAIM OF PRIORITY

This application is a non-provisional application and claims priority to U.S. Provisional Patent Application No. 62/060,813 having a priority date of Oct. 7, 2014, the contents of which are hereby incorporated by reference.

### FIELD OF THE EMBODIMENTS

This invention relates to a computer implemented method for improving health care management and compliance by reducing the rate of adverse outcomes through the calculation of a Coordination Risk score, identification of a predominant prescriber, and assessment of compliance with prescribed maintenance medications.

### BACKGROUND OF THE EMBODIMENTS

There is a need to provide those responsible for the healthcare of others with information at a population and an individual level to assist in the provision of care and improve compliance measures while being fiscally responsible. Commercial insurers, Medicare Advantage programs, Exchange programs, Accountable Care Organizations (ACO), Independent Physicians Associations (IPA), self-insured employer groups, Third Party administrators, hospitals, emergency rooms and among others, are in need of assistance to determine where to focus their efforts with respect to care management and compliance with prescribed medications. There is a need to provide them with key data including an analysis of factors which could impede coordination efforts and impact both the health outcomes of patients and the cost of providing care. Research has shown that three factors associated with a patient's prescription drug use: 1) Polyprescriber (multiple prescribers; 2) Polypharmacy (multiple drugs), and 3) Therapeutic complexity (multiple drugs influencing different body chemistry), strongly influence a healthcare organization's ability to coordinate their care effectively. In addition, identification of a predominant prescriber provides care managers a focal point for communication and intervention. Finally, evidence of potential non-compliance with prescription drug therapies points to patient behaviors that influence clinical outcomes of treatment efforts. Key data sources include health insurers, PBMs (Pharmacy Benefit Managers), Health Insurance Exchange (HIX), Electronic Medical Records (EMR), Pharmacy data consolidators and health information networks such as SureScripts and WebMD. In addition, hospitals and their emergency rooms utilize data from third party vendors such as a Health Insurance Exchange (HIX), Pharmacy Benefit Manager (PBM), or Electronic Medical Record (EMR) and provide patient reports to the attending physician. Similarly, Health Care Providers utilize data from third party vendors and provide patient reports to physicians. Therefore, there is a need for a computer implemented system that can provide accurate and timely data to assess and predict Coordination Risk.

Coordination Risk is a measure of the probability that, owing to a lack of coordination among caregivers, a patient/member will experience an adverse outcome such as a visit to the Emergency Room, an inpatient hospital admission or observation stay, or a progression in the acuity of a medical condition. Some events of this nature will occur randomly and be unavoidable. However, research has shown that certain factors can be quantified that inhibit coordination among caregivers and increase the probability of an adverse outcome. The present computer implemented method assesses component factors independently and then combines said factors into a single Coordination Risk score. The two additional measures provided in parallel with the coordination risk score provide guidance for care managers' initiatives to reduce risk.

The Coordination Risk score is described along a five-unit continuum, from “very low” to “very high.” Early identification of patients/members with high Coordination Risk scores gives users an opportunity to intervene and potentially reduce the rate of adverse outcomes. The present invention system identifies factors which independently and in combination present challenges to optimal care coordination and therefore contributes to the risk of an adverse outcome. These factors include but are not limited to Polypharmacy; Polyprescriber and Therapeutic Complexity.

### Review of Related Technology:

U.S. Pat. No. 7,523,042 teaches a patient tracking and identification of patient care deficiencies system which includes: Identification of populations of patients with the a similar condition, Alerts of overdue care necessary to monitor status of condition (e.g., blood pressure monitoring for hypertension), Alerts identifying patient candidates for screening and/or preventive tests or measures, Alerts of overdue laboratories to monitor medication safety (e.g., PT/INR testing on patients receiving warfarin to determine appropriate dose changes), Stratification of patients based on risk of a negative healthcare outcomes (e.g., ordering patients by highest risk for heart attack); Benchmarking of healthcare provider performance with automated feedback and Timely provision of relevant medical evidence and guidelines. These three functions are provided automatically and continuously, using data extraction methodology from an EMR.

U.S. Pat. No. 8,799,030 discloses methods and systems for disease management care coordination. In one embodiment, claim data associated with a prescription drug is integrated with additional health data from a plurality of data feeds received from a client device, a care coordination device, and a health management vendor device. The integrated data is targeted to identify a member. The integrated data associated with the member is transmitted to a patient evaluator device. The integrated data is updated using additional member information received from the patient evaluator device. Other methods and systems are described.

U.S. Pat. No. 8,700,433 discloses a computer-implemented method for profiling medical claims to assist health care managers in determining the cost-efficiency and service quality of health care providers. The method allows an objective means for measuring and quantifying health care services. An episode treatment group (ETG) is a patient classification unit, which defines groups that are clinically homogenous (similar cause of illness and treatment) and statistically stable. The ETG grouper methodology uses service or segment-level claim data as input data and assigns each service to the appropriate episode. The program identifies concurrent and recurrent episodes, flags records, creates new groupings, shills groupings for changed conditions, selects the most recent claims, resets windows, makes a determination if the provider is an independent lab and continues to collect information until an absence of treatment is detected.

U.S. Pat. No. 6,370,511 discloses a computer-implemented method of grouping pharmaceutical claims data, comprising the steps of: (a) reading pharmaceutical claims data, input as at least one of a plurality of data records, into a computer memory; (b) validating each of the at least one of a plurality of data records for a valid drug code; (c) reading at least one pre-defined relationship between the valid drug code in the validated at least one of a plurality of data records and pre-defined episode treatment categories; and (d) grouping the validated at least one of a plurality of data records to an episode treatment group based upon the pre-defined relationship read in step (c).

U.S. Pat. No. 8,719,051 teaches an evaluative software tool to support the assessment of health care-related technologies during development to facilitate making critical decisions for an optimized research, development and commercialization plan is provided. The software tool includes the ability to measure, weight, and integrate the critical factors that come into play in the development of a risk/benefit profile of a technology relative to its competitors, benchmarked around clinical trial measures, in order to determine its development and commercialization success.

U.S. Patent Publication No. 20110099024 teaches a system generally comprising: a) a computer database for maintaining personal and medical records of a patient; b) means for remotely accessing said database to at least one provider of medical care for said patient; c) records of personal and medical information entered into said database by said provider or electronically downloaded; d) an algorithm program for artificial intelligence relating a diagnosed medical condition of said patient and at least one medical care action relating to said patient; e) means for communication between medical providers simultaneously or at different times; f) means of web cam communication; g) means of identifying laboratory values or test results not in normal range; h) means of scheduling appointments; i) means of scanning documents; j) means of billing; and k) printable patient information.

U.S. Patent Publication No. US 20130197942 discloses a dynamic risk management system for use in providing remote medical management services is disclosed and described. The system includes a database and at least one processor that is programmed to calculate a dynamic risk score for each patient in a plurality of patients. The dynamic risk score is calculated continuously and receives real time data related to the patients. Based on each patient's risk score, patient care resources are dynamically allocated to the patient population and/or treatment decisions are made for the patients.

U.S. Patent Publication No. US 20130191140 relates to methods and systems for coordinating care for a care recipient among one or more caregivers. Caregivers can organize and assign tasks related to a diagnosis, illness, condition, behavior, living condition, etc. of a care recipient. Interactive systems and methods are disclosed that allow multiple caregivers to accomplish a plurality of tasks, generate care related reports, and/or monitor medication compliance.

U.S. Patent Publication No. 20110191115 teaches a computer software program operable to provide an ongoing and supported care coordination approach in which nurses and physician extenders work to actively link a patient with the best provider of care for the patient's unique condition. The program works with the patient to ensure compliance with physician orders, and assists with appointments, transportation and the delivery of medical services.

U.S. Patent Publication No. US 20050065816A discloses a method for generating a visual compliance display comprising: providing a plurality of different compliance obligations; providing a first user interface having a first input area, a second input area and a status area; receiving first input data relating to an information and associated with a compliance obligation, said first input data provided to said first input area; automatically determining a first scalable level of compliance in dependence upon compliance obligations satisfied by said first input data; automatically displaying within said status area of said first user interface an indication based on said first scalable level of compliance; receiving second input data relating to another information and associated with a compliance obligation, said second input data provided to said second input area; automatically determining a second scalable level of compliance in dependence upon compliance obligations satisfied by said first input data and said second input data; and automatically displaying within said status area of said first user interface an indication based on said second scalable level of compliance.

An example of related art is U.S. Pat. No. 8,099,306 entitled Pharmacy Episodes of Care. U.S. Pat. No. 8,099,306 discloses a computer-implemented method for grouping, categorizing, and profiling pharmaceutical claims data to assist health care managers in determining (a) medication treatment experience, outcomes, and medication compliance behaviors of patients and (b) appropriate drug prescribing, medication coordination, and cost-efficiency of health care providers. However, the art described above addresses all of the issues that the present invention does.

Various systems are known in the art. However, their function and means of operation are substantially different from the present invention. Such systems fail to provide accurate and timely data to assess—and predict coordination risk. At least one embodiment of this invention is presented in the drawings below and will be described in more detail herein.

### SUMMARY OF THE EMBODIMENTS

The present invention comprises a computer implemented method for quantifying a risk associated with medical and health care, the steps of which comprise; memory that stores computer executable instructions; a processor, communicatively coupled to the memory that facilitates execution of the computer executable instructions, said instructions comprising; calculating, via a processor, a first value; the first value equal to the medications prescribed to a plurality of individuals in a specified population; calculating, via a processor, a second value; the second value equal to the prescribers of the first value of the plurality of individuals in a specified population; calculating, via a processor, a third value; the third value equal to the associated therapeutic classifications of the first value of the plurality of individuals in a specified population; calculating, via a processor, an average value of each first, second and third value; comparing, via a processor, the average value of each first, second and third value against a fourth value; the fourth value equal to a single individual of the first, second and third value; transforming, via a processor, the first, second, third and fourth values into a fifth value, calculating, via a processor, the fifth value which corresponds to a degree of risk of adverse outcomes related to the healthcare management of an individual.

The method further comprises where the first value is indicative of a polypharmaceutical measurement. The first value further comprises the average number of unique prescribed pharmaceuticals for a plurality of individuals in a specified population. The second value is indicative of a polyprescriber measurement. The second value comprises the average number of prescribers for a plurality of individuals in a specified population. The third value is indicative of a therapeutic complexity. The third value is the average number of therapeutic classification for a plurality of individuals in a specified population. The fifth value is indicative of a coordination risk score. The method also comprises a prediction engine. The first, second, third, fourth and fifth values are dynamically weighted. The prediction engine transforms the first second and third value into a prediction of the fifth value. The method also comprises a sixth value which is equal to a plurality of initiating prescriptions for unique drugs by a single prescriber for the individual. The sixth value is indicative of a predominant prescriber. The method further comprises a defined time in expected usage or non-usage of a drug by the individual. The defined time further comprises noncompliance or non-adherence by the individual of a prescribed medication therapy.

The present invention also comprises a system for quantifying a risk associated with medical and health care comprising; memory that stores computer executable instructions; a processor, communicatively coupled to the memory that facilitates execution of the computer executable instructions, said instructions comprising; calculating, via a processor, a first value; the first value equal to the medications prescribed to a plurality of individuals in a specified population; calculating, via a processor, a second value; the second value equal to the prescribers of the first value of the plurality of individuals in a specified population; calculating, via a processor, a third value; the third value equal to the associated therapeutic classifications of the first value of the plurality of individuals in a specified population; calculating, via a processor, an average value of each first, second and third value; comparing, via a processor, the average value of each first, second and third value against a fourth value; the fourth value equal to a single individual of the first, second and third value; transforming, via a processor, the first, second, third and fourth values into a fifth value, calculating, via a processor, the fifth value which corresponds to a degree of risk of adverse outcomes related to the healthcare management of an individual.

It is an object of the present invention where the demographic of the individual is a factor to be recorded.

It is an object of the present invention where the line of business of the individual is a factor to be recorded.

It is an object of the present invention where a predetermined amount of time corresponding to the care of an individual is a factor to be adjusted.

### BRIEF DESCRIPTION OF THE DRAWINGS

FIG. 1 shows an illustrative view of the architecture of the system of the invention.

FIG. 2 shows an illustrative view of the flow of data through the system of the invention.

FIG. 3 shows an illustrative view of a summary report of the system of the invention.

FIG. 4 shows an illustrative view of a patient profile of the system of the invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Three factors independently and in combination present challenges to optimal care coordination and therefore contribute to the risk of an adverse outcome. These are “Polypharmacy,” “Polyprescriber,” and “Therapeutic Complexity.”

Polypharmacy Concept:

In its simplest form, this is a count of the different prescription drugs provided to a patient/member. First, the number of drugs is generally a good indication that a patient has multiple conditions, so to some degree, the measure is a proxy for illness burden. In addition, the larger the number of drugs, particularly if they are prescribed by different practitioners who do not communicate with one another, the more likely it is that problems will emerge. In this calculation, the present invention identifies each drug uniquely but without regard to differences such as packaging and brand. It is not a count of prescription volume as it also does not count refills.

In the present invention, a client's claim file for prescription drugs to count the number of prescriptions for “Unique Drugs” (i.e., it excludes refills, packaging, brand, and dosage variations for the same drugs, so it is not a count of overall volume of prescriptions) associated with each person in that client's member file. Next, the software calculates the mean (average) number of prescriptions/person for that client. Because a client applies the present invention separately to each Line of Business (elderly, nonelderly, Medicaid, private/commercial, Medicare, etc.) owing to differences in scope of benefits, age distributions, and so on, this strategy is valid across client types. The calculated “client mean” serves as the “client standard” for subsequent calculation of a five-unit scale for polypharmacy status as follows:
very low signifies greater than 1 standard deviation below the client mean
low signifies less than ½ the standard deviation to 1 standard deviation below the client mean
average signifies ½ the standard deviation below the mean to 1 standard deviation above the mean
high signifies greater than 1 standard deviation above the mean to 3 standard deviations above the mean
very high signifies greater than 3 standard deviations above the mean

Polyprescriber Concept:

This is a count of the different prescribers, using prescriber National Provider Identifier (NPI) or Drug Enforcement Agency (DEA) identifier, recorded on each drug claim in a patient's member file. When a patient provides information to the present inventions system, the system allows an individual prescriber's ID to be linked to a Clinic or Group Practice ID. The present invention may utilize the group/clinic identifier in place of the individual practitioner identifier. This method takes into account that many patients utilize clinic/group where several of the practitioners could authorize a prescription or refill because they share in patient care and employ a uniform electronic health record (EHR). Physicians are unanimous in their concern that multiple prescribers complicate care management and increase risks of adverse events.

In the present invention, a client's claim file for prescription drugs to count the number of prescribers (using NPI or DEA identifiers) associated with each script for each person in a client's member file. Next, the software calculates the mean (average) number of prescribers/member for that client. Because a client applies the PrescienceRx software separately to each Line of Business (elderly, nonelderly, Medicaid, private/commercial, Medicare, etc.) owing to differences in scope of benefits, age distributions, and so on, this strategy is valid across client types. The calculated “client mean” serves as the “client standard” for subsequent calculation of a five-unit scale for polyprescriber status as follows:
very low signifies greater than 1 standard deviation below the client mean
low signifies less than ½ the standard deviation to 1 standard deviation below the client mean
average signifies ½ the standard deviation below the mean to 1 standard deviation above the mean
high signifies greater than 1 standard deviation above the mean to 3 standard deviations above the mean
very high signifies greater than 3 standard deviations above the mean

Therapeutic Complexity:

This is a count of the different therapeutic classifications or classes (TCs) represented by the individual prescription drugs. To some extent, the number of different TCs is a proxy for multiple morbidities, or total illness burden. The larger the number of TCs involved in a patient's care, the greater the number of body systems/functions being addressed pharmacologically. The present invention incorporates an industry standard classification system (MediSpan, owned by Wolters-Kluwer) to map each drug's National Drug Code (NDC) to a therapeutic class.

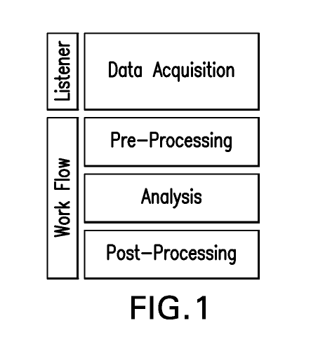

FIG. 1 shows the method and system Being comprised of three components: a computation engine called the Data Factory, a high-speed parallel database, and a mobile application. The calculation of the system described here are performed by the Data Factory. FIG. 1 shows the high level view of the components of the Data Factory. The Data Factory is comprised of four components: Data Acquisition, Pre-processing, Analysis, and Post-processing. The Data Acquisition component is driven by a listener which waits for incoming data then starts the appropriate processing when files are received. The remaining three components execute a series of computations guided by the PrescienceRx workflow. The Analysis component is a massively parallel processing big data engine that converts raw data such as patient, physician, and prescription data into predictive and descriptive demographics and statistics. Polypharmy, Polyprescriber, Therapeutic Complexity, and the Coordination Risk score are computed here. Two additional calculations are performed by the Analysis software component. The first identifies a predominant prescriber for each patient which is the prescriber that initiates the largest number of prescriptions for a drug, ignoring refills, changes in dosage, or packaging/form differences. The second calculation reviews selective “critical” use prescriptions for maintenance medications to test for potential non-compliance. This computed and analyzed data is retained within the systems database portion (PrescienceRx-DB) and presented to users through the user interface (PrescienceRx-UX).

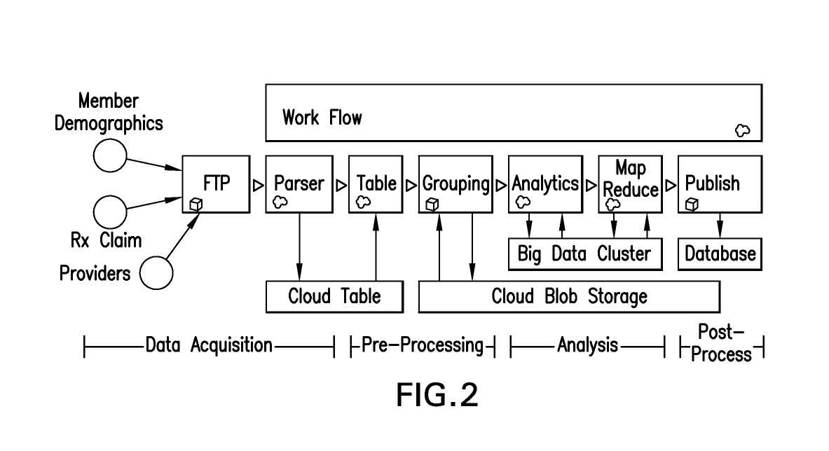

Calculating the Coordination Risk score and associated component statistics requires health plan membership and prescription data. This data is parsed, processed, and analyzed through a sequence of software logic. As shown in FIG. 2, the data is processed to yield means and standard deviations which are compiled and applied to individual patients to derive the Polypharmacy, Polyprescriber, Therapeutic Complexity, and Coordination Risk metrics as well as identification of predominant prescriber and potential non-compliance. As shown in FIG. 2, information and data flows through a series of activities in the Computation Engine. First it is copied to the cloud and parsed into formats that can be manipulated by the software. Once formatted, further analytics are added to the data by a predictive model. Finally, the solution computes Polypharmacy, Polyprescriber, Therapeutic Complexity, and Coordination Risk via algorithms that are implemented on a software framework that is able to process massive amounts of data across a distributed cluster of processors or stand-alone computers within a network such as Hive and MapReduce.

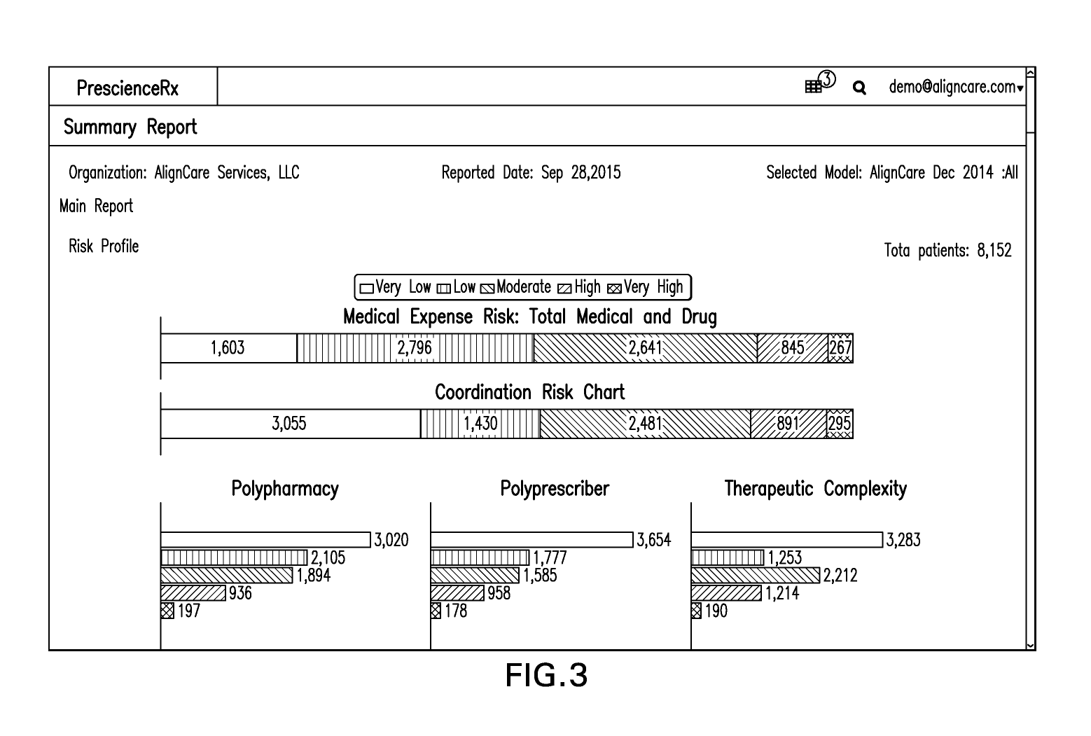

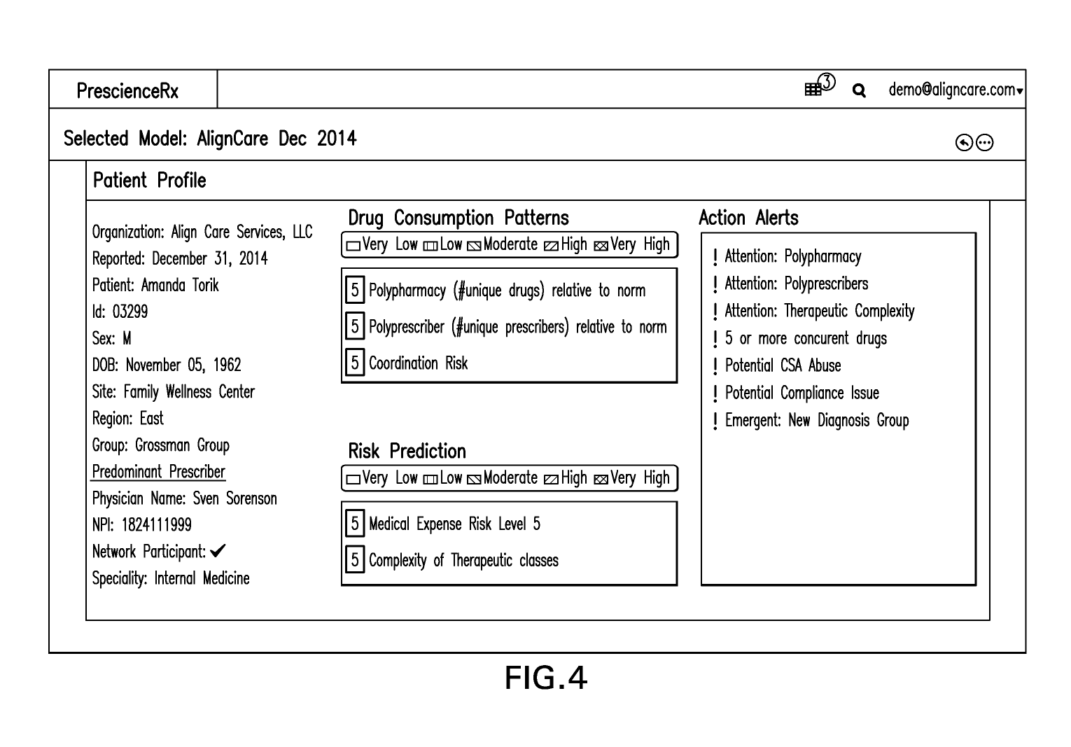

The metrics may be presented in color-coded visualization on a web based dashboard in the form of summary reports (as shown in FIG. 3) and patient profiles (as shown in FIG. 4). The summary report contains information such as the predicted expense of the total cost of medical and drug treatment and the Coordination Risk score. The summary report also contains information on a patient's drug consumption patterns as well as metrics related to prescriber metrics and the other factors related to Coordination Risk. The Summary Report illustrates how the results and values of the system in processing Polypharmacy, Polyprescriber, and Therapeutic Complexity are aggregated and processed. In the Summary Report, the analytics may be displayed via bar chart visualization. These values may also be displayed in other forms such as pie charts or tables.

The patient profile page contains similar information related to the patient. The Patient Profile illustrates how the results and values of the system are presented for an Individual. In the Patient Profile report, the analytics are displayed as color coded values. These values may be displayed in other forms such as thermometers or gauges. A patient's predominant prescriber is identified in the detail section of the patient report which shows every script that has been filled and every prescriber. The patient report also contains an “alert” feature. The alert is triggered when the software has identified potential non-compliance with a critical use maintenance drug, and provides in the detail section, a full history of the identified drug or drugs so identified.

In the present invention, a client's claim file for prescription drugs to map each NDC to a “therapeutic class” and associates the count of different TCs with each person in a client's member file. Note that any difference in brand/package/or dosage is identified within the same therapeutic class, and this measure only counts the number of different therapeutic classes, not the volume of drugs by TC. Next, the system calculates the mean (average) number of TCs/member for that client. Because a client applies the system's components separately to each Line of Business (elderly, nonelderly, Medicaid, private/commercial, Medicare, 5 etc.) owing to differences in scope of benefits, age distributions, and so on, this strategy is valid across client types. The calculated “client mean” serves as the “client standard” for subsequent calculation of a five-unit scale for therapeutic complexity status (therapeutic complexity) as follows:
very low signifies greater than 1 standard deviation below the client mean
low signifies less than ½ the standard deviation to 1 standard deviation below the client mean
average signifies ½ the standard deviation below the mean to 1 standard deviation above the mean
high signifies greater than 1 standard deviation above the mean to 3 standard deviations above the mean
very high signifies greater than 3 standard deviations above the mean

Coordination Risk Score: Each of the component measures is a quantification of challenges for care coordination and predictive to some degree of the risks of adverse outcomes if coordination is impaired. The system of the present invention harnesses the power of each measure to create a total “Coordination Risk” score. At present, each measure carries the same weight. However, in another embodiment, each measure may carry varying or different weights and may be used as a predictor for the Coordination's Risk score as well as a predictor for the Coordination Risk score's component factors (Polypharmacy, Polyprescriber and Therapeutic Class). In another embodiment, the Coordination Risk score and its component factors may be used independently as well as in combination to predict an Emergency Room (ER or ED, emergency ROOM/department) visit or an In-Patient (Hospital) Admission or observation stay. In another embodiment, the system will comprise a prediction engine which may result in the component factors having a different weighing algorithm for classifying patients along the Coordination Risk spectrum from very low to very high.

In the present invention, the system assigns a value for each status indicator for each component measure of Coordination Risk to derive the composite CR Score as shown in Table 1:

TABLE 1

| Coordination | | | | | |
|---|---|---|---|---|---|
| Risk | | | | | |
| component | Very Low | Low | Average | High | Very High |
| Polypharmacy | 1 | 2 | 3 | 4 | 5 |
| Polyprescriber | 1 | 2 | 3 | 4 | 5 |
| Therapeutic | 1 | 2 | 3 | 4 | 5 |
| Complexity | | | | | |

The lowest possible value is 3; the highest possible value is 15. If a patient is “very low” for polypharmacy (value=1), for polyprescriber (value=1), and for therapeutic complexity (value=1), that patient's coordination risk score is 3 (1+1+1) and will be shown in the output as Very Low Coordination Risk. If a patient is “low” for polypharmacy (value=2), is “high” for polyprescriber (value=5), and “average” for therapeutic complexity (value=3), that patient's coordination risk score is 10 (2+5+3) and will be shown in the output as Average Coordination Risk. The range of possible values is from 3 to 15, and at present, they are grouped into a final Coordination Risk score for each patient as shown in Table 2. Different groupings may be made in future based on research outcomes exploring differential weights for the components of Coordination Risk.

TABLE 2

| Coordination Risk Score | Aggregate Component Values |
|---|---|
| Very Low | 3 or 4 |
| Low | 5 or 6 |
| Average (Moderate) | 7-10 |
| High | 11 or 12 |
| Very High | 13-15 |

A Coordination Risk score is assigned to each client-covered member for the time period under review. A Coordination Risk is recalculated each time a client updates the system's reports. The system displays changes/trends in each patient's Coordination Risk score for each client-defined time period. For example, a client could identify patterns for patients in a care management program compared to patients who are not participating in care management.

Predominant Prescriber (PP) is identifies for each patient by summing the number of unique prescriptions for each prescriber, based on the NPI, and assigning predominant prescriber status based on plurality. In the event of an equal number of prescriptions, PP is conferred on the prescriber associated with the pharmacy-based medical group (RxMG) with the greatest potential for general high total medical expense as determined by the Johns Hopkins University Methodology, adjusted clinical groups (ACG), and pharmacy model.

Non-compliance/non-adherence is identified for each patient by longitudinal review of prescriptions for maintenance medications, emphasizing a sub group classified as “critical use,” meaning that an interruption in routine use is associated with relatively short term adverse consequences. To be considered non-compliant, a patient must have had no script/refill for a period equal to one and a half times of that days' supply associated with the most recent script that has been filled, for a minimum of two consecutive months. In addition, the present invention system confirms that no therapeutic equivalent or treatment alternative drug is present before designating a patient as potentially non-compliant/non-adherent.

The objective of the present invention is to have the most accurate solutions to stratify populations, identify individuals with a high probability of an acute event in the near future and help determine or predict a course of action. Such determinations and prediction are fundamental to improving and reducing the cost of care. In another embodiment of the present invention, additional data points and perspectives may be added to the system. These may include adding data such as health screens to lab data, further research on the interrelationship between drug classes and/or comorbidities, and the introduction of machine learning to improve the predictive models. In another embodiment of the present invention, data from an electronic medical record, beyond prescription data may be mined as a real time data source. In another embodiment of the present invention, interoperability with core systems and mobile devices may be included. In another embodiment of the present invention, actionable data may be accessed, processed, and determined with increased speed.

Typically, a user or users, which may be people or groups of users and/or other systems, may engage information technology systems (e.g., computers) to facilitate operation of the system and information processing. In turn, computers employ processors to process information and such processors may be referred to as central processing units (“CPU”). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations. One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the present invention may be connected to and/or communicate with Entities such as, but not limited to: one or more users from user input devices; peripheral devices; an optional cryptographic processor device; and/or a communications network. For example, the present invention may be connected to and/or communicate with users operating client device(s), including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™ HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The present invention may be based on computer systems that may comprise, but are not limited to, components such as: a computer systemization connected to memory.

Computer Systemization

A computer systemization may comprise a clock, central processing unit (“CPU(s)” and/or “processor(s)” (these terms are used interchangeable throughout the disclosure unless noted to the contrary)), a memory (e.g., a read only memory (ROM), a random access memory (RAM), etc.), and/or an interface bus, and most frequently, although not necessarily, are all interconnected and/or communicating through a system bus on one or more (mother)board(s) having conductive and/or otherwise transportive circuit pathways through which instructions (e.g., binary encoded signals) may travel to effect communications, operations, storage, etc. Optionally, the computer systemization may be connected to an internal power source; e.g., optionally the power source may be internal. Optionally, a cryptographic processor and/or transceivers (e.g., ICs) may be connected to the system bus. In another embodiment, the cryptographic processor and/or transceivers may be connected as either internal and/or external peripheral devices via the interface bus I/O. In turn, the transceivers may be connected to antenna(s), thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing the controller of the present invention to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like. The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the present invention and beyond through various interfaces. Should processing requirements dictate a greater amount speed and/or capacity, distributed processors (e.g., Distributed embodiments of the present invention), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the present invention may be achieved by implementing a microcontroller such as CAST's R8051XC2 microcontroller; Intel's MCS 51 (i.e., 8051 microcontroller); and/or the like. Also, to implement certain features of the various embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the component collection (distributed or otherwise) and/or features of the present invention may be implemented via the microprocessor and/or via embedded components; e.g., via ASIC, coprocessor, DSP, FPGA, and/or the like. Alternately, some implementations of the present invention may be implemented with embedded components that are configured and used to achieve a variety of features or signal processing.

Depending on the particular implementation, the embedded components may include software solutions, hardware solutions, and/or some combination of both hardware/software solutions. For example, features of the present invention discussed herein may be achieved through implementing FPGAs, which are a semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the features of the present invention. A hierarchy of programmable interconnects allow logic blocks to be interconnected as needed by the system designer/administrator of the present invention, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the present invention may be developed on regular FPGAs and then migrated into a fixed version that more resembles ASIC implementations. Alternate or coordinating implementations may migrate features of the controller of the present invention to a final ASIC instead of or in addition to FPGAs. Depending on the implementation all of the aforementioned embedded components and microprocessors may be considered the “CPU” and/or “processor” for the present invention.

Power Source

The power source may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell is connected to at least one of the interconnected subsequent components of the present invention thereby providing an electric current to all subsequent components. In one example, the power source is connected to the system bus component. In an alternative embodiment, an outside power source is provided through a connection across the I/O interface. For example, a USB and/or IEEE 1394 connection carries both data and power across the connection and is therefore a suitable source of power.

Interface Adapters

Interface bus(ses) may accept, connect, and/or communicate to a number of interface adapters, conventionally although not necessarily in the form of adapter cards, such as but not limited to: input output interfaces (I/O), storage interfaces, network interfaces, and/or the like. Optionally, cryptographic processor interfaces similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces may accept, communicate, and/or connect to a number of storage devices such as, but not limited to: storage devices, removable disc devices, and/or the like.

Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces may accept, communicate, and/or connect to a communications network. Through a communications network, the controller of the present invention is accessible through remote clients (e.g., computers with web browsers) by users. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed embodiments of the present invention), architectures may similarly be employed to pool, load balance, and/or otherwise increase the communicative bandwidth required by the controller of the present invention. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces may be used to engage with various communications network types. For example, multiple network interfaces may be employed to allow for the communication over broadcast, multicast, and/or unicast networks.

Input Output interfaces (I/O) may accept, communicate, and/or connect to user input devices, peripheral devices, cryptographic processor devices, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices often are a type of peripheral device (see below) and may include: card readers, dongles, finger print readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices, such as other components of the cooling chest system, including temperature sensors, ice dispensers (if provided) and the like may be connected and/or communicate to I/O and/or other facilities of the like such as network interfaces, storage interfaces, directly to the interface bus, system bus, the CPU, and/or the like. Peripheral devices may be external, internal and/or part of the controller of the present invention. Peripheral devices may also include, for example, an antenna, audio devices (e.g., line-in, line-out, microphone input, speakers, etc.), cameras (e.g., still, video, webcam, etc.), drive motors, ice maker, lighting, video monitors and/or the like.

Cryptographic units such as, but not limited to, microcontrollers, processors, interfaces, and/or devices may be attached, and/or communicate with the controller of the present invention. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+MB/s of cryptographic instructions; VLSI Technology's 33 MHz 6868; and/or the like.

Memory

Generally, any mechanization and/or embodiment allowing a processor to affect the storage and/or retrieval of information is regarded as memory. However, memory is a fungible technology and resource, thus, any number of memory embodiments may be employed in lieu of or in concert with one another. It is to be understood that the controller of the present invention and/or a computer systemization may employ various forms of memory. For example, a computer systemization may be configured wherein the functionality of on-chip CPU memory (e.g., registers), RAM, ROM, and any other storage devices are provided by a paper punch tape or paper punch card mechanism; of course such an embodiment would result in an extremely slow rate of operation. In a typical configuration, memory will include ROM, RAM, and a storage device. A storage device may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

Component Collection

The memory may contain a collection of program and/or database components and/or data such as, but not limited to: operating system component(s) (operating system); information server component(s) (information server); user interface component(s) (user interface); Web browser component(s) (Web browser); database(s); mail server component(s); mail client component(s); cryptographic server component(s) (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device, they may also be loaded and/or stored in memory such as: peripheral devices, RAM, remote storage facilities through a communications network, ROM, various forms of memory, and/or the like.

Operating System

The operating system component is an executable program component facilitating the operation of the controller of the present invention. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millennium/NT/Vista/XP (Server), Palm OS, and/or the like. The operating system may be one specifically optimized to be run on a mobile computing device, such as iOS, Android, Windows Phone, Tizen, Symbian, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the controller of the present invention to communicate with other entities through a communications network. Various communication protocols may be used by the controller of the present invention as a subcarrier transport mechanism for interaction, such as, but not limited to: multicast, TCP/IP, UDP, unicast, and/or the like.

Information Server

An information server component is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective-) C (++), C# and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTTP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the controller of the present invention based on the remainder of the HTTP request. For example, a request such as http://123.124.125.126/myInformation.html might have the IP portion of the request “123.124.125.126” resolved by a DNS server to an information server at that IP address; that information server might in turn further parse the http request for the “/myInformation.html” portion of the request and resolve it to a location in memory containing the information “myInformation.html.” Additionally, other information serving protocols may be employed across various ports, e.g., FTP communications across port, and/or the like. An information server may communicate to and/or with other components in a component collection, including itself, and/or facilities of the like. Most frequently, the information server communicates with the database of the present invention, operating systems, other program components, user interfaces, Web browsers, and/or the like.

Access to the database of the present invention may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the present invention. In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the present invention as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millennium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

Web Browser

A Web browser component is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, User Agents, and/or the like from the enabled nodes of the present invention. The combined application may be nugatory on systems employing standard Web browsers.

Mail Server

A mail server component is a stored program component that is executed by a CPU. The mail server may be a conventional Internet mail server such as, but not limited to sendmail, Microsoft Exchange, and/or the like. The mail server may allow for the execution of program components through facilities such as ASP, ActiveX, (ANSI) (Objective-) C (++), C# and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the present invention.

Access to the mail of the present invention may be achieved through a number of APIs offered by the individual Web server components and/or the operating system.

Also, a mail server may contain, communicate, generate, obtain, and/or provide program component, system, user, and/or data communications, requests, information, and/or responses.

Mail Client

A mail client component is a stored program component that is executed by a CPU. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

Cryptographic Server

A cryptographic server component is a stored program component that is executed by a CPU, cryptographic processor, cryptographic processor interface, cryptographic processor device, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), Digital Signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MD5, which is a one way hash function), passwords, Rivest Cipher (RC5), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the present invention may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MD5 hash to obtain a unique signature for an digital audio file. A cryptographic component may communicate to and/or with other components in a component collection, including itself, and/or facilities of the like. The cryptographic component supports encryption schemes allowing for the secure transmission of information across a communications network to enable the component of the present invention to engage in secure transactions if so desired. The cryptographic component facilitates the secure accessing of resources on the present invention and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

The Database of the Present Invention

The database component of the present invention may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the database of the present invention may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

In one embodiment, the database component includes several tables. A user (e.g., operators and physicians) table may include fields such as, but not limited to: user_id, ssn, dob, first_name, last_name, age, state, address_firstline, address_secondline, zipcode, devices_list, contact_info, contact_type, alt_contact_info, alt_contact_type, and/or the like to refer to any type of enterable data or selections discussed herein. The user's table may support and/or track multiple Entity accounts. A Client's table may include fields such as, but not limited to: user_id, client_id, client_ip, client_type, client_model, operating_system, os_version, app_installed_flag, and/or the like. An Apps table may include fields such as, but not limited to: app_ID, app_name, app_type, OS_compatibilities_list, version, timestamp, developer_ID, and/or the like. A Parameter table may include fields including the foregoing fields, or additional ones such as cool_start_time, cool_preset, cooling_rate, and/or the like. A Routines table may include a plurality of sequences which may include fields such as, but not limited to, and in the case of Cooling Temperature sequences: sequence_type, sequence_id, flow_rate, avg_water_temp, cooling_time, pump_setting, pump_speed, pump_pressure, power_level, temperature_sensor_id_number, temperature_sensor_location, and/or the like.

In one embodiment, user programs may contain various user interface primitives, which may serve to update the platform of the present invention. Also, various accounts may require custom database tables depending upon the environments and the types of clients the system of the present invention may need to serve. It should be noted that any unique fields may be designated as a key field throughout. In an alternative embodiment, these tables have been decentralized into their own databases and their respective database controllers (i.e., individual database controllers for each of the above tables). Employing standard data processing techniques, one may further distribute the databases over several computer systemizations and/or storage devices. Similarly, configurations of the decentralized database controllers may be varied by consolidating and/or distributing the various database components. The system of the present invention may be configured to keep track of various settings, inputs, and parameters via database controllers.

The many elements of the present invention make it unique in the field. Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1) A computer implemented method for quantifying a risk associated with medical and health care, the steps of which comprise;
memory that stores computer executable instructions;
a processor, communicatively coupled to the memory that facilitates execution of the computer executable instructions, said instructions comprising;
calculating, via a processor, a first value; said first value equal to the medications prescribed to a plurality of individuals in a specified population;
calculating, via a processor, a second value; said second value equal to the prescribers of the first value of the plurality of individuals in a specified population;
calculating, via a processor, a third value; said third value equal to the associated therapeutic classifications of the first value of the plurality of individuals in a specified population;
calculating, via a processor, an average value of each first, second and third value;
comparing, via a processor, the average value of each first, second and third value against a fourth value; said fourth value equal to a single individual of the first, second and third value;
transforming, via a processor, the first, second, third and fourth values into a fifth value,
calculating, via a processor, said fifth value which corresponds to a degree of risk of adverse outcomes related to the healthcare management of an individual.

2) The method of claim 1, where the first value is indicative of a polypharmaceutical measurement.

3) The method of claim 1, wherein the first value further comprises the average number of unique prescribed pharmaceuticals for a plurality of individuals in a specified population.

4) The method of claim 1, wherein the second value is indicative of a polyprescriber measurement.

5) The method of claim 1, wherein the second value comprises the average number of prescribers for a plurality of individuals in a specified population.

6) The method of claim 1, wherein the third value is indicative of a therapeutic complexity.

7) The method of claim 1, wherein the third value is the average number of therapeutic classification for a plurality of individuals in a specified population.

8) The method of claim 1, wherein a demographic of the individual is a factor to be recorded.

9) The method of claim 1, wherein a line of business of the individual is a factor to be recorded.

10) The method of claim 1, wherein the fifth value is indicative of a coordination risk score.

11) The method of claim 1, wherein the first, second, third, fourth and fifth values are dynamically weighted.

12) The method of claim 1, further comprising a prediction engine.

13) The method of claim 13, wherein the prediction engine transforms the first second and third value into a prediction of the fifth value.

14) The method of claim 1, wherein a predetermined amount of time corresponding to the care of an individual is a factor to be adjusted.

15) The method of claim 1, further comprising a sixth value; said sixth value equal to a plurality of initiating prescriptions for unique drugs by a single prescriber for the individual.

16) The method of claim 15, wherein the sixth value is indicative of a predominant prescriber.

17) The method of claim 1, further comprising a defined time in expected usage or non usage of a drug by the individual.

18) The method of claim 18, where the defined time further comprises noncompliance or non-adherence by the individual of a prescribed medication therapy.

19) A system for quantifying a risk associated with medical and health care comprising;
memory that stores computer executable instructions;
a processor, communicatively couples to the memory that facilitates execution of the computer executable instructions, said instructions comprising;
calculating, via a processor, a first value; said first value equal to the medications prescribed to a plurality of individuals in a specified population;
calculating, via a processor, a second value; said second value equal to the prescribers of the first value of the plurality of individuals in a specified population;
calculating, via a processor, a third value; said third value equal to the associated therapeutic classifications of the first value of the plurality of individuals in a specified population;
calculating, via a processor, an average value of each first, second and third value;
comparing, via a processor, the average value of each first, second and third value against a fourth value; said fourth value equal to a single individual of the first, second and third value;
transforming, via a processor, the first, second, third and fourth values into a fifth value,
calculating, via a processor, said fifth value which corresponds to a degree of risk of adverse outcomes related to the healthcare management of an individual.
